(19) United States
(12) Patent Application Publication
(10) Pub. No.: US 2013/0035309 A1
(43) Pub. Date: Feb. 7, 2013

(54) OPHTHALMIC SEALANT COMPOSITION AND METHOD FOR USE

(71) Applicants: Lisa A. Butterick, Swedesboro, NJ (US); Henry Keith Chenault, Hockessin, DE (US); Grant L. Vincent, Wilmington, DE (US)

(72) Inventors: Lisa A. Butterick, Swedesboro, NJ (US); Henry Keith Chenault, Hockessin, DE (US); Grant L. Vincent, Wilmington, DE (US)

(73) Assignee: Actamax Surgical Materials ,LLC, Berkeley, CA (US)

(21) Appl. No.: 13/641,217

(22) PCT Filed: Apr. 12, 2011
(86) PCT No.: PCT/US2011/032104

§ 371 Date: Oct. 15, 2012

Related U.S. Application Data
(60) Provisional application No. 61/323,475, filed on Apr. 13, 2010.

(51) Int. Cl. A61K 31/721; A61P 27/02

(52) U.S. Cl. 514/59

(57) ABSTRACT

A polymeric hydrogel sealant specifically formulated to seal ophthalmic wounds is provided. The sealant is formed by mixing two aqueous solutions. The first aqueous solution comprises an oxidized dextran having a specific average molecular weight range and oxidation level and the second aqueous solution comprises a 4-arm polyethylene glycol having two primary amine groups at the end of substantially every arm. A kit and method for sealing an ophthalmic wound using the hydrogel sealant is also provided.

### CROSS-REFERENCE TO RELATED APPLICATION

This patent application is a continuation-in-part application of PCT application No. PCT/US2011/032104 filed Apr. 12, 2011, claiming priority under 35 U.S.C. §119 from U.S. Provisional Application Ser. No. 61/323,475, filed Apr. 13, 2010.

### FIELD OF THE INVENTION

The invention relates to the field of medical sealants and liquid bandages. More specifically, the invention relates to a polymeric hydrogel sealant specifically formulated to seal ophthalmic wounds caused by trauma or surgery.

### BACKGROUND OF THE INVENTION

Ophthalmic wounds result from trauma such as corneal lacerations, or from surgical procedures such as vitrectomy procedures, cataract surgery, LASIK surgery, glaucoma surgery, and corneal transplants. These wounds are typically sealed using sutures; however, the use of sutures has some drawbacks. Specifically, the placement of sutures inflicts trauma to the site, especially when multiple passes are required. Sutures may also serve as a site for infection and may lead to inflammation and vascularization, thereby increasing the chances of scarring. Additionally, the use of sutures may lead to uneven healing, resulting in astigmatism. For some procedures such as sealing corneal cataract incisions, many surgeons prefer sutureless, self-sealing incisions because of the drawbacks of using sutures. However, sutureless incisions may leak and are points of potential ingress into the anterior chamber by foreign bodies or contaminating fluids, which may cause complications such as endophthalmitis.

A potential alternative to sutures for sealing ophthalmic wounds is the use of ophthalmic sealants. Various types of sealants have been proposed for sealing ophthalmic wounds. For example, cyanoacrylates have been proposed as having utility as a corneal sealant. However, the disadvantage is that cyanoacrylates can be toxic due to the formation of formaldehyde. Additionally, cyanoacrylate sealants are rigid and thus cause discomfort, detach from the eye in as little as one day, and do not degrade readily. The use of fibrin sealants to seal ophthalmic wounds has also been proposed; however, fibrin sealants usually lack the required adhesive strength, pose a risk of viral infection, may inhibit wound healing, and may result in an increased incidence of inflammation.

Several types of hydrogel tissue sealants have been developed, which have improved adhesive and cohesive properties. These hydrogels are generally formed by reacting a component having nucleophilic groups with a component having electrophilic groups which are capable of reacting with the nucleophilic groups of the first component, to form a crosslinked network via covalent bonding. Some types of these hydrogel sealants have been reported to be useful for ophthalmic applications (Rhee, et al., U.S. Patent Application Publication No. 2004/0235708; and Grinstaff, et al., WO 2006/031358). However, ophthalmic sealants having improved properties are still needed.

Kodokian, et al. (copending and commonly owned U.S. Patent Application Publication No. 2006/0078536) describe hydrogel tissue adhesives formed by reacting an oxidized polysaccharide with a water-dispersible, multi-arm polyether amine. These adhesives provide improved adhesion and cohesion properties, crosslink readily at body temperature, maintain dimensional stability initially, do not degrade rapidly, and are nontoxic to cells and non-inflammatory to tissue. However, for use specifically for ophthalmic applications, a sealant should possess an additional combination of certain properties to be most effective. Specifically, the sealant should have low cytotoxicity to corneal endothelial cells, have low swell, and seal reliably for the required period of time depending on the application and then degrade away. Additionally, the sealant should not cause patient discomfort or interfere with vision.

Therefore, the problem to be solved is to provide a polymeric hydrogel sealant with improved characteristics for use in sealing ophthalmic wounds caused by trauma or surgery.

### SUMMARY OF THE INVENTION

Ophthalmic sealants comprising polymeric hydrogels are provided.

In one embodiment the disclosure provides a kit comprising:

a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and

b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

In another embodiment, the disclosure provides a composition comprising the reaction product of:

a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and

b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

In another embodiment, the disclosure provides a method of sealing an ophthalmic wound comprising applying to the wound

a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and

b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

### DETAILED DESCRIPTION OF THE INVENTION

Disclosed herein is a polymeric hydrogel sealant composition specifically formulated to provide the combination of properties necessary for use in ophthalmic applications. The polymeric hydrogel sealant is formed by mixing two aqueous solutions. The first aqueous solution comprises an oxidized dextran having a weight-average molecular weight of about 8,500 to about 11,500 Daltons and an equivalent weight per aldehyde group of about 130 to about 165 Daltons, and the second aqueous solution comprises a 4-arm polyethylene glycol substantially each arm of which has two primary amine groups at its end and having a number-average molecular weight of about 9,000 Daltons to about 11,000 Daltons.

The polymeric hydrogel sealant disclosed herein possesses the following properties which make it well suited for sealing a corneal incision resulting from cataract surgery. Specifically, the polymeric hydrogel sealant has very low cytotoxicity to corneal endothelial cells, has low swell, and seals reliably for 3 days, then degrades, as described in Examples 4-10. The very low cytotoxicity to endothelial cells is particularly important because corneal endothelial cells do not replicate. It is expected that the polymeric hydrogel sealant formulation can be tuned to seal reliably for the period of time required for other ophthalmic applications. In fact, no corneal irritation was observed in animals treated with the sealant, as described in Example 10. The sealant should not cause patient discomfort or interfere with vision because it is a clear, soft, pliant hydrogel. The sealant disclosed herein may be used to seal ophthalmic wounds such as sclerotomy incisions created during a vitrectomy procedure, corneal incisions resulting from cataract surgery, LASIK flaps, and corneal lacerations. Additionally, the sealant may also be useful for sealing bleb leaks after glaucoma surgery and for sealing the cornea after a corneal transplant.

### DEFINITION OF TERMS

The following definitions are used herein and should be referred to for interpretation of the claims and the specification.

The terms “oxidized dextran” and “dextran aldehyde” are used interchangeably herein to refer to dextran which has been reacted with an oxidizing agent to introduce aldehyde groups into the molecule.

The term “equivalent weight per aldehyde group” refers to the average molecular weight of the oxidized dextran divided by the number of aldehyde groups introduced in the molecule.

The terms “% by weight” and “wt %” as used herein refer to the weight percent of solute relative to the total weight of the solution.

The term “Mₙ” as used herein means number-average molecular weight.

The term “M_{w}” as used herein means weight-average molecular weight.

The term “hydrogel” as used herein refers to a water-swellable polymeric matrix, consisting of a three-dimensional network of macromolecules held together by covalent crosslinks that can absorb a substantial amount of water to form an elastic gel.

The term “wound”, as used herein, refers to an anatomical disruption of the eye caused by trauma or surgery.

Further, the meaning of abbreviations used is as follows: “min” means minute(s), “h” means hour(s), “sec” means second(s), “d” means day(s), “mL” means milliliter(s), “L” means liter(s), “μL” means microliter(s), “cm” means centimeter(s), “mm” means millimeter(s), “μm” means micrometer(s), “mol” means mole(s), “mmol” means millimole(s), “g” means gram(s), “mg” means milligram(s), “meq” means milliequivalent(s), the designation “10K” means that a polymer molecule possesses a number-average molecular weight of 10 kiloDaltons, “¹H NMR” means proton nuclear magnetic resonance spectroscopy, “M” means molar concentration, “Pa” means pascal(s), “kPa” means kilopascal(s), “PEG” means polyethylene glycol, “MW” means molecular weight, “EW” means equivalent weight, “D” means density, “bp” means boiling point.

A reference to “Aldrich” or a reference to “Sigma” means the noted chemical or ingredient was obtained from Sigma-Aldrich, St. Louis, Mo.

### First Aqueous Solution

The first aqueous solution comprises an oxidized dextran containing aldehyde groups, having a weight-average molecular weight of about 8,500 to about 11,500 Daltons and an equivalent weight per aldehyde group of about 130 to about 165 Daltons.

Dextran suitable for use herein has a weight-average molecular weight before oxidation of about 8,500 to about 11,500 Daltons, and is available commercially from companies such as Sigma-Aldrich (St. Louis, Mo.) and Pharmacosmos (Holbaek, Denmark). Typically, commercial preparations of dextran are a heterogeneous mixture having a distribution of different molecular weights, as well as a variable degree of branching, and are characterized by various molecular weight averages, for example, the weight-average molecular weight (M_{w}), or the number-average molecular weight (Mₙ), as is known in the art.

The dextran is oxidized to introduce aldehyde groups using methods known in the art. The dextran may be oxidized using any suitable oxidizing agent, including but not limited to, periodates, hypochlorites, ozone, peroxides, hydroperoxides, persulfates, and percarbonates. In one embodiment, the dextran is oxidized by reaction with sodium periodate, for example as described by Mo, et al. (J. Biomater. Sci. Polymer Edn. 11: 341-351, 2000). The amount of periodate used is adjusted to provide a degree of oxidation of about 50%, as described below in the General Methods Section of the Examples. It should be understood that the degree of oxidation obtained will be within a range, due to small experimental variations and experimental error. For example, the degree of oxidation of the dextran will typically be about 45% to about 55%. The oxidation does not alter the average molecular weight of the dextran significantly. Therefore, the weight-average molecular weight of the oxidized dextran useful as described herein is about 8,500 to about 11,500 Daltons. The oxidized dextran with an oxidation level of about 50% has an equivalent weight per aldehyde group of about 130 to about 165 Daltons.

In one embodiment, the oxidized dextran is prepared by the method described by Cohen, et al. (copending and commonly owned International Patent Application Publication No. WO 2008/133847), as described in detail in the General Methods Section of the Examples below. This method of making an oxidized polysaccharide, which comprises a combination of precipitation and separation steps to purify the oxidized polysaccharide formed by oxidation of the polysaccharide with periodate, provides an oxidized dextran with very low levels of iodine-containing species.

The degree of oxidation, also referred to herein as the oxidation conversion, of the oxidized dextran may be determined using methods known in the art. For example, the degree of oxidation may be determined using the method described by Hofreiter, et al. (Anal Chem. 27: 1930-1931, 1955). In this method, the amount of alkali consumed per mole of dialdehyde in the oxidized dextran, under specific reaction conditions, is determined by a pH titration. Alternatively, the degree of oxidation of the dextran may be determined using nuclear magnetic resonance (NMR) spectroscopy.

The first aqueous solution can be prepared by adding the appropriate amount of the oxidized dextran to water to give the desired concentration, specifically, about 15 wt % to about 30 wt %, more particularly about 20 wt % to about 25 wt %, and more particularly about 25 wt %.

For use on living tissue, it is preferred that the first aqueous solution comprising the oxidized dextran be sterilized to prevent infection. Any suitable sterilization method known in the art that does not adversely affect the ability of the oxidized dextran to form an effective sealant may be used, including, but not limited to, electron beam irradiation, gamma irradiation, or ultra-filtration through a 0.2 μm pore membrane.

The first aqueous solution may further comprise a colorant to aid in the visualization of the solution during application. Suitable colorants include dyes, pigments, and natural coloring agents. Examples of suitable colorants include, but are not limited to, FD&C and D&C colorants, such as FD&C Violet No. 2, FD&C Blue No. 1, D&C Green No. 6, D&C Green No. 5, D&C Violet No. 2; and natural colorants such as beetroot red, canthaxanthin, chlorophyll, eosin, saffron, and carmine. In one embodiment the colorant is FD&C Blue No. 1.

The first aqueous solution may optionally include at least one pH modifier to adjust the pH of the solution. Suitable pH modifiers are well known in the art. The pH modifier may be an acidic or basic compound. Examples of acidic pH modifiers include, but are not limited to, carboxylic acids, inorganic acids, and sulfonic acids. Examples of basic pH modifiers include, but are not limited to, hydroxides, alkoxides, nitrogen-containing compounds other than primary and secondary amines, and basic carbonates and phosphates.

The first aqueous solution may also comprise at least one pharmaceutical drug or therapeutic agent. Suitable drugs and therapeutic agents for ophthalmic applications are well known in the art and include, but are not limited to, antimicrobial agents such as antibiotics (e.g., macrolides, fluoroquinolones, and aminoglycosides); anti-inflammatory agents such as corticosteroids (e.g., prednisone, fluorometholone, and dexamethasone), and combinations thereof.

### Second Aqueous Solution

The second aqueous solution comprises a 4-arm polyethylene glycol (PEG) wherein substantially each arm has two primary amine groups at its end (also referred to herein as 4-arm polyethylene glycol amine or 4-arm PEG amine). As used herein, the phrase “substantially each arm has two primary amine groups at its end” means that at least 50% of the arms have two primary amine groups at their ends, more particularly at least 70% of the arms have two primary amine groups at their ends, more particularly at least 80% of the arms have two primary amine groups at their ends, and more particularly at least 90% of the arms have two primary amine groups at their ends. The remaining arms may have single primary amine groups or hydroxyl groups at their ends. Other end groups are also possible, provided that they do not make the PEG amine cytotoxic or interfere with crosslinking to form the hydrogel. In one embodiment, about 100% of the arms of the 4-arm polyethylene glycol have two primary amine groups at their end (also referred to herein as 4-arm polyethylene glycol octaamine or 4-arm PEG octaamine). The 4-arm polyethylene glycol wherein substantially each arm has two primary amine groups at its end has a number-average molecular weight of about 9,000 Daltons to about 11,000 Daltons, more particularly about 10,000 Daltons.

A 4-arm PEG amine can be prepared using the method described by Arthur (copending and commonly owned International Patent Application No. PCT/US07/24393, WO 2008/066787), in which a molecule containing two primary amine groups is added to the ends of a 4-arm PEG polyol. The starting 4-arm PEG polyol having a number-average molecular weight of about 9,000 Daltons to about 11,000 Daltons is available commercially from companies such as Shearwater Polymers Inc, (Huntsville, Ala.). For example, the 4-arm PEG polyol may be reacted with thionyl chloride in a suitable solvent such as toluene to give the chloride derivative, which is subsequently reacted with tris(2-aminoethyl)amine to give the 4-arm PEG octaamine, as described in detail in the General Methods of the Examples herein below.

It should be recognized that the 4-arm PEG polyol can be a somewhat heterogeneous mixture having a distribution of arm lengths, resulting in a distribution of molecular weights. Therefore, the resulting 4-arm PEG amine will also have a distribution of arm lengths, resulting in a distribution of molecular weights.

To prepare the second aqueous solution, the appropriate amount of the 4-arm PEG amine is added to water to give the desired concentration, specifically, about 15 wt % to about 45 wt %, more particularly about 25 wt % to about 40 wt %, more particularly about 30 wt % to about 35 wt %, and more particularly about 30 wt %.

In one embodiment, the concentration of the oxidized dextran in the first aqueous solution is about 25 wt % and the concentration of the 4-arm PEG amine in the second aqueous solution is about 30 wt %.

For use on living tissue, it is preferred that the second aqueous solution comprising the 4-arm PEG amine be sterilized to prevent infection. Any of the methods described above for sterilizing the first aqueous solution may be used.

The second aqueous solution may further comprise a colorant to aid in the visualization of the solution during application. Any of the colorants described above for the first aqueous solution may be used. In one embodiment at least one of the first aqueous solution or the second aqueous solution further comprises a colorant.

The second aqueous solution may further comprise a pharmaceutical drug or therapeutic agent, such as described above for the first aqueous solution.

Additionally, it may be desirable to include at least one acidic pH modifier to lower the pH of the second aqueous solution to prevent eye irritation. Examples of acidic pH modifiers include, but are not limited to, carboxylic acids, inorganic acids, and sulfonic acids. In one embodiment, at least one acidic pH modifier is added to the second aqueous solution so that the pH of the hydrogel resulting from the combination of the first and second aqueous solutions has a pH in the range of about 6.5 to about 8.0.

### Ophthalmic Sealant Kit

In one embodiment, the invention provides an ophthalmic sealant kit comprising a first aqueous solution comprising an oxidized dextran, as described above, and a second aqueous solution comprising a 4-arm PEG amine, as described above. Each of the aqueous solutions may be contained in any suitable vessel, such as a vial or a syringe barrel. The kit may further comprise a suitable delivery device to deliver the two aqueous solutions to the site of the wound, as described below. The kit may also comprise a set of instructions describing the use of the kit.

### Method of Sealing an Ophthalmic Wound

The polymeric hydrogel sealant disclosed herein may be used to seal an ophthalmic wound resulting from trauma or surgery. For example, the sealant may be used to seal ophthalmic wounds such as sclerotomy incisions created during a vitrectomy procedure, corneal incisions resulting from cataract surgery, LASIK flaps, bleb leaks after glaucoma surgery, and for sealing the cornea after a corneal transplant. All of these surgical procedures are well known to skilled ophthalmic surgeons. After the surgical procedure, the first and second aqueous solutions are applied to the wound as described below to seal the incision. Additionally, the sealant may be used to seal ophthalmic wounds caused by trauma such as corneal lacerations.

In one embodiment, the first aqueous solution and the second aqueous solution are applied to the wound simultaneously without premixing. The compositions of the first and second aqueous solutions are specifically formulated to form the hydrogel sealant via diffusional mixing after application of the two aqueous solutions to the wound site. This eliminates complications associated with the use of a delivery device having a static mixer to premix the solutions before application to the site, for example, clogging of the mixer. However, the two aqueous solutions may be premixed before application if desired. The sealant may be applied within the wound (i.e., between the edges of the incision) or overlaying the wound.

In one embodiment, the first aqueous solution and the second aqueous solution are applied to the wound in a 1:1 volume ratio. However, other volume ratios may also be used, for example volume ratios of the first aqueous solution to the second aqueous solution of 1:3, 1:2, 1:1.5, 2:1, 1.5:1, or any convenient volume ratio may be used.

The first and second aqueous solutions can be applied to the wound in a number of ways. For example, the two aqueous solutions may be coated on the sides of a scalpel blade or keratome, one solution on each side of the blade, to apply them to the wound site when the site is ready for closure. Alternatively, a double barrel delivery device may be used to deliver the two aqueous solutions simultaneously to the wound without premixing the solutions. The delivery device should be capable of delivering minute quantities (e.g., about 50 to about 3000 nanoliters) of each of the two aqueous solutions to the wound site. A delivery device that allows premixing of the two solutions just prior to application may also be employed. Suitable delivery devices may be made by miniaturizing double barrel delivery devices described in the art (see for example Miller, et al., U.S. Pat. No. 4,874,368; and Redl, U.S. Pat. No. 6,620,125) to deliver small volumes.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

### Reagents

Dextran having a M_{w} of 8,500 to 11,500 Daltons (referred to herein as “D10”) was purchased from Sigma-Aldrich (St Louis, Mo.). The 8-arm PEG (Mₙ=10,000, referred to herein as “8-arm PEG 10K”), having eight arms, each terminated by a hydroxyl group, was purchased from NOF Corp. (Tokyo, Japan). The 4-arm PEG (Mₙ=10,000, referred to herein as “4-arm PEG 10K”), having four arms, each terminated by a hydroxyl group, was purchased from Shearwater Polymers Inc., (Huntsville, Ala.; Lot 03616). Sodium periodate (99% purity, CAS No. 7790-28-5) was purchased from Acros Organics (Morris Plains, N.J.). All other reagents were obtained from Sigma-Aldrich (St. Louis, Mo.) unless otherwise noted.

### Preparation of Oxidized Dextran (D10-50):

Dextran aldehyde was made by oxidizing dextran in aqueous solution with sodium metaperiodate. An oxidized dextran having an average molecular weight of about 10,000 Da, an oxidation conversion of about 50% (i.e., about half of the glucose rings in the dextran polymer were oxidized to dialdehydes) and an equivalent weight (EW) per aldehyde group of about 150 was prepared from dextran having a weight-average molecular weight of 8,500 to 11,500 Daltons (Sigma) by the method described by Cohen, et al. (copending and commonly owned International Patent Application Publication No. WO 2008/133847). A typical procedure is described below.

A 20-L reactor equipped with a mechanical stirrer, addition funnel, internal temperature probe, and nitrogen purge was charged with 1000 g of the dextran and 9.00 L of de-ionized water. The mixture was stirred at ambient temperature to dissolve the dextran and then cooled to 10 to 15° C. To the cooled dextran solution was added over a period of an hour, while keeping the reaction temperature below 25° C., a solution of 1000 g of sodium periodate dissolved in 9.00 L of de-ionized water. Once all the sodium periodate solution was added, the mixture was stirred at 20 to 25° C. for 4 more hours. The reaction mixture was then cooled to 0° C. and filtered to clarify. Calcium chloride (500 g) was added to the filtrate, and the mixture was stirred at ambient temperature for 30 min and then filtered. Potassium iodide (400 g) was added to the filtrate, and the mixture was stirred at ambient temperature for 30 min. A 3-L portion of the resulting red solution was added to 9.0 L of acetone over a period of 10 to 15 min with vigorous stirring by a mechanical stirrer during the addition. After a few more minutes of stirring, the agglomerated product was separated from the supernatant liquid. The remaining red solution obtained by addition of potassium iodide to the second filtrate was treated in the same manner as above. The combined agglomerated product was broken up into pieces, combined with 2 L of methanol in a large stainless steel blender, and blended until the solid became granular. The granular solid was recovered by filtration and dried under vacuum with a nitrogen purge. The granular solid was then hammer milled to a fine powder. A 20-L reactor was charged with 10.8 L of de-ionized water and 7.2 L of methanol, and the mixture was cooled to 0° C. The granular solid formed by the previous step was added to the reactor and the slurry was stirred vigorously for one hour. Stirring was discontinued, and the solid was allowed to settle to the bottom of the reactor. The supernatant liquid was decanted by vacuum, 15 L of methanol was added to the reactor, and the slurry was stirred for 30 to 45 min while cooling to 0° C. The slurry was filtered in portions, and the recovered solids were washed with methanol, combined, and dried under vacuum with a nitrogen purge to give about 600 g of the oxidized dextran, which is referred to herein as D10-50.

The degree of oxidation of the product was determined by proton NMR to be about 50% (equivalent weight per aldehyde group=150). In the NMR method, the integrals for two ranges of peaks were determined, specifically, —O₂CHx- at about 6.2 parts per million (ppm) to about 4.15 ppm (minus the HOD peak) and —OCHx- at about 4.15 ppm to about 2.8 ppm (minus any methanol peak if present). The calculation of oxidation level was based on the calculated ratio (R) for these areas, specifically, R=(OCH)/(O₂CH).

$$\% oxidation = 100 - \frac{300 \times ( R - 1 )}{3 + 2 \times R}$$

### Preparation of 8-Arm PEG 10K Octaamine (P8-10-1):

Eight-arm PEG 10K octaamine (Mₙ=10 kDa) was synthesized using the two-step procedure described by Chenault in co-pending and commonly owned U.S. Patent Application Publication No. 2007/0249870. In the first step, the 8-arm PEG 10K octachloride was made by reacting thionyl chloride with the 8-arm PEG 10K octaol. In the second step, the 8-arm PEG 10K octachloride was reacted with aqueous ammonia to yield the 8-arm PEG 10K octaamine. A typical procedure is described below.

The 8-arm PEG 10K octaol (Mₙ=10000, SunBright HGEO-10000, NOF Corp., 1000 g) was dissolved in 1.5 L of toluene under an atmosphere of nitrogen in a 4-L glass reaction vessel equipped with a stirrer, reflux condenser and distillation head. The mixture was dried azeotropically by distillative removal of about 500 mL of toluene under reduced pressure (13 kPa, pot temperature 65° C.). The mixture was brought back to atmospheric pressure with nitrogen, and thionyl chloride (233 mL) was added to the mixture over 10 min, keeping the pot temperature below 85° C. After the addition of thionyl chloride was complete, the mixture was heated to 85° C. and stirred at 85° C. for 4 hours. Excess thionyl chloride and most of the toluene was removed by vacuum distillation (2 kPa, pot temperature 40-60° C.). Two successive 500-mL portions of toluene were added and evaporated under reduced pressure (2 kPa, bath temperature 60° C.) to complete the removal of thionyl chloride. The pressure was reduced to 0.7-0.9 kPa, and distillation was continued with a pot temperature of 85° C. for 60-90 minutes to complete the removal of toluene.

Proton NMR results: ¹H NMR (500 MHz, DMSO-d₆) δ 3.71-3.69 (m, 16H), 3.67-3.65 (m, 16H), 3.50 (s, ˜800H).

While the product was still warm, it was dissolved in 1 L of de-ionized water and discharged from the reaction vessel.

The aqueous solution of 8-arm PEG 10K octachloride prepared above was combined with 16 L of concentrated aqueous ammonia (28 wt %) in a 5-gallon stainless steel pressure vessel equipped with a stirrer, and the atmosphere was replaced with nitrogen. The vessel was sealed, and the mixture was heated at 60° C. for 48 hours. The mixture was cooled to 40° C. and sparged with dry nitrogen (2 L/min) for 18-24 hours to drive off ammonia. The nitrogen flow was stopped, and the mixture was stirred under vacuum (2 kPa) for 2 hours at 40° C. The remaining solution was passed through 5.0 kg of strongly basic anion exchange resin (Purolite® A-860, The Purolite Co., Bala-Cynwyd, Pa.) in the hydroxide form packed a 30 inch-long×6 inch-outer diameter column. The eluant was collected, and two 7-L portions of de-ionized water were passed through the column and also collected. The combined aqueous solutions were concentrated under reduced pressure (2 kPa, bath temperature 60° C.) and then dried further at 60° C./0.3 kPa to give about 942 g of the 8-arm PEG 10K octaamine, referred to herein as P8-10−1, as a colorless waxy solid.

### Preparation of 8-Arm PEG 10K Hexadecaamine (P8-10-2):

Eight-arm PEG 10K hexadecamine (Mₙ=10 kDa, average of 16 primary amine groups per polymer molecule) was synthesized using the two-step procedure described by Arthur, et al. in co-pending and commonly owned International Patent Application Publication No. WO 2008/066787. In the first step, the 8-arm PEG 10K octamesylate was made by reacting the 8-arm PEG 10K octaol with methanesulfonyl chloride in the presence of triethylamine. In the second step, the 8-arm PEG 10K octamesylate was reacted in water with tris(2-aminoethyl)amine to yield the 8-arm PEG 10K hexadecamine. A typical procedure is described below.

Triethylamine (8.8 mL) was added to a solution of 40 g of the 8-arm PEG 10K octaol (Mₙ=10000, SunBright HGEO-10000, NOF Corp.) in 200 mL of CH₂Cl₂ under a blanket of nitrogen. The mixture was cooled with stirring in an ice-water bath. Methanesulfonyl chloride (4.8 mL) was added dropwise to the stirred reaction mixture at 0° C. (CAUTION: EXOTHERM). When the addition of methanesulfonyl chloride was complete, the ice-water bath was removed, and the reaction was stirred at room temperature overnight. The reaction volume was reduced to 80 mL by rotary evaporation and transferred to a separatory funnel, where it was washed gently three times with 60 mL portions of 1.0 M aqueous potassium dihydrogen phosphate, once with 60 mL of 1 M aqueous potassium carbonate, and once with 60 mL of water. The CH₂Cl₂ layer was dried over of MgSO₄, filtered, and concentrated by rotary evaporation to afford syrup (yield of 39.8 g (94%)).

A solution of 30 g of 8-arm PEG 10K octamesylate in 149 mL of water was added to a solution of 149 mL of tris(2-aminoethyl)amine in 149 mL of water, and the mixture was stirred at room temperature overnight. Aqueous sodium bicarbonate (10 wt %, 150 mL) was added to the reaction mixture, which was then extracted three times with 180-mL portions of CH₂Cl₂. The combined organic layer was dried over MgSO₄, filtered, and concentrated by rotary evaporation. The resulting clear syrup was precipitated in 500 mL of ether and cooled in an ice bath. The white solid was collected by filtration and dried under high vacuum overnight (yield of 24.4 g (86%)). The 8-arm PEG 10K hexadecamine product is referred to herein as P8-10-2.

### Preparation of 4-Arm PEG 10K Octaaamine (P4-10-2):

Four-arm PEG 10K octaamine (Mₙ=10 kDa, average of 8 primary amine groups per polymer molecule) was synthesized using the two-step procedure described by Arthur, et al. in co-pending and commonly owned International Patent Application Publication No. WO 2008/066787. In the first step, the 4-arm PEG 10K octachloride was made by reacting the 4-arm PEG 10K octaol with thionyl chloride in the presence of triethylamine. In the second step, the 4-arm PEG 10K octachloride was reacted in water with tris(2-aminoethyl)amine to yield the 4-arm PEG 10K octaamine. A typical procedure is described below.

A solution of 50.0 g (20 mmol OH; OH EW=2500) 4-arm PEG 10K (Mₙ=10,000; Shearwater Polymers Inc, Lot 03616) and 0.1 mL of dimethylacetamide in 100 mL of toluene was heated to 80° C. in a 250-mL round bottom flask with condenser and drying tube to form a solution. The solution was cooled to 60° C. and stirred as 5 mL of thionyl chloride (8.2 g; 68 mmol; MW=118.97; D=1.63; bp: 79° C.) was added. A gel initially formed due to formation of sulfite ester crosslinks but soon dispersed to a solution as the sulfite bonds reacted with HCl and cleaved to sulfur dioxide and PEG chloride end groups.

The mixture was stirred at 60° C. for 22 hours and then was suction-filtered through Celite® diatomaceous earth (World Minerals, Lompoc, Calif.) to remove haze. The clear filtrate was rotovapped to remove thionyl chloride and about 50 mL of toluene was added, followed by addition of 1.0 mL (25 mmol) of methanol to scavenge any remaining thionyl chloride. The solution was then added with stirring to 300 mL of hexane as the PEG chloride product coated out on the bottom of the flask. The hexane was decanted off and replaced with 200 mL of fresh hexane and the polymer was broken up with a spatula and magnetically stirred at room temperature. Over a couple hours of stirring the 4-arm star PEG 10K tetrachloride product became powdered; it was suction-filtered, washed once with 100 mL of hexane and suctioned dry under a nitrogen blanket to yield 46.3 g.

Proton nuclear magnetic resonance spectroscopy (¹H NMR) (CDCl₃): 3.41 ppm (s, 1.6H); 3.64 (s, 220H); 3.75 (t, J=6.0 Hz, 2.2H); overlaps with spinning side bands so the integral is high. The pentaerythritol core CH₂ at 3.41 ppm always integrated low; perhaps it relaxes differently.

A 0.53-g sample of 4-arm star PEG 10K tetrachloride sample was heated at 100° C. with 3 mL of acetic anhydride and 3 mL of pyridine for 3 hours. The solution was rotovapped and then held in hot water under a nitrogen stream under vacuum for 1 hour ¹H NMR (CDCl₃): 3.41 ppm (s, 1.6H); 3.64 (s, 220H); 3.75 (t, J=6.0 Hz, 2.2H); 4.22 (t, J=4.9 Hz, 0.04H). These results indicate that about 8% of the ends in the PEG chloride product were OH.

A solution of 12.0 g (5 mmol Cl; Cl EW=2500) of the 4-arm star PEG 10K tetrachloride in 40 mL of water was stirred rapidly in a 100° C. oil bath as 30 mL (30 g; 205 mmol) of tris(2-aminoethyl)amine (MW=146.2; D=0.98; TCI America, Portland, Oreg.; #T1243) was added. The resulting mixture was stirred at 100° C. for 21 hours. Then, 0.5 mL (9 mmol) of 50 wt % sodium hydroxide solution was added and the resulting solution was extracted with 3×40 mL of dichloromethane. The combined extracts were dried with magnesium sulfate, rotovapped to 30 mL and precipitated into 400 mL of ether with stirring. The suspension was stirred in an ice bath for 20 min and the resulting white precipitate was suction-filtered, washed with 50 mL of diethyl ether on the funnel, and dried under nitrogen to yield 9.9 g (78%) of the 4-arm star PEG 10K amine as a white powder; herein referred to as P4-10-2.

¹H NMR (CDCl₃): 2.51 ppm (t, 3.4H); 2.60 (t, 1.8H); 2.71 (t, 1.7H); 2.75 (t, 3.4H); 2.80 (t, 1.8H); 3.41 ppm (s, 1.6H); 3.64 (s, 220H); 3.75 CH₂Cl (gone). The peaks were sharp. These results indicate that the ends of the PEG arms are approximately 90% functional, the remainder being OH.

### Examples 1-3

### Ex-Vivo Burst Testing of a Sealed Incision in the Eye

The following Examples demonstrate the burst strength of a sealed incision in enucleated porcine eyes using different sealant formulations. A 3.2 mm clear corneal incision (non-self sealing) was made 2-3 mm from the corneal limbal margin of enucleated porcine eyes, obtained from SiouxPreme Packing Co. (Sioux Center, Iowa), approximately 24 hours after death, to mimic the incision made during cataract surgery. After placing the incision, an air bubble (approximately 1 cm in diameter) was placed into the anterior chamber of each eye to maintain appropriate incision alignment and prevent leaking of intraocular fluid from the wound during sealant application, and the corneal surface was wiped dry with a surgical sponge. Then, a first aqueous solution and a second aqueous solution (see Table 1) were applied to the incision simultaneously without premixing using a dual component micro delivery device (fabricated in-house). The micro delivery device was a double barrel syringe made from two tubes, each having a plunger. To apply the two aqueous solutions to the wound, the tip of the delivery device was placed on the outer edge of the incision and the solutions were deposited. Each of the tubes held approximately 1.5 μL of one of the aqueous solutions, which resulted in application of 2 to 3 μL of total sealant. After application, the resulting mixture was allowed to gel for approximately 3 min.

TABLE 1
Sealant Formulations

| Example | First Aqueous Solution | Second Aqueous Solution |
|---|---|---|
| 1 | D10-50 | P4-10-2 |
| | 25 wt % | 30 wt % |
| 2, Comparative | D10-50 | P8-10-2 |
| | 20 wt % | 30 wt % |
| 3, Comparative | D10-50 | P8-10-1 |
| | 25 wt % | 60 wt % |

After the incision was sealed, the eyes were placed in a burst pressure test apparatus (one eye at a time) and the burst pressure measurements were made as follows. A syringe pump (Syringe Infusion Pump, Harvard Apparatus Model 22, South Natick, Mass.) with an inline pressure gauge (Omega Model No. DPG5000L, Range: 15G Z PK F16) was used to fill the anterior chamber of each eye with BSS® (Balanced Salt Solution; Alcon Laboratories, Fort Worth, Tex.) contained in two 50 mL syringes (Becton Dickinson 50 mL syringe, Luer-Lok Tip), and the maximum intraocular pressure prior to burst was measured. A tube with a needle (Becton Dickinson Precision Glide, 30G1) fastened to one end, was attached to the pressure port of the syringe pump. The pump needle was then inserted into the anterior chamber of the eye. Additional pressure was generated in the anterior chamber by actuating the syringe pump to perfuse at a rate of 100 μL/min. The needle was inserted into the anterior chamber of the eye directly opposite (i.e. 180°) from the incision location. Pressure rose within the anterior chamber of the eye as fluid volume increased. At the first sight of a leak, the pressure was recorded as the burst pressure of the incision. Results of the burst pressure testing are summarized in Table 2.

TABLE 2
Burst Pressure Results

| | Mean Burst Pressure | Standard | Performance |
|---|---|---|---|
| Example | (mm Hg) | Deviation | Rating |
| 1 | 82.27 | 17.16 | Pass |
| | (11.0 kPa) | (2.29 kPa) | |
| 2. Comparative | 84.28 | 26.65 | Pass |
| | (11.2 kPa) | (3.55 kPa) | |
| 3, Comparative | 108.12 | 27.65 | Pass |
| | (14.4 kPa) | (3.69 kPa) | |

Sealant performance was deemed acceptable when the mean burst pressure was above 70 mm Hg (9.3 kPa), identified from literature references to be the maximum burst pressure of an incision placed in a human eye and closed with one suture. By this criterion, all the sealant formulations tested displayed sufficient burst pressure to function as an ophthalmic sealant.

### Examples 4-6

### In Vitro Biocompatibility Testing

### NIH3T3 Fibroblast Assay

These Examples demonstrate the concentration at which various multi-arm PEG amines become toxic to NIH3T3 fibroblasts in an in vitro assay.

Stock solutions of the multi-arm PEG amines, P8-10-2, P8-10-1 and P4-10-2, were prepared in Dulbecco's Modified Eagles Medium (DMEM), obtained from Invitrogen Corp. (Carlsbad, Calif.), at a final concentration of 100 mg/mL. The solutions were sterile filtered and placed in an incubator at 37° C., 5% CO₂. The PEG amine stock solutions were allowed to equilibrate to pH 7.0, and then were diluted using DMEM containing 10% calf serum to final concentrations of 10, 5.0, 2.5, 1.0, 0.5, and 0.1 mg/mL.

NIH3T3 cells, obtained from ATCC (Manassas, Va.), were cultured in T75 flasks to 80% confluence, trypsinized, and suspended in DMEM containing 10% calf serum to a final concentration of 1×10⁵ cells/mL. To a 96 well cell culture, tissue treated plate, 100 μL of the stock cell solution was added to each well resulting in a final concentration of 10,000 cells/well. Cells were grown overnight in an incubator at 37° C., 5% CO₂ to allow for attachment and growth. The following day, all cell culture medium was removed and the cells were dosed with 100 μL of the PEG amine solutions. After 24 hours of exposure to the test sample, cellular viability was assayed using the WST-8 reagent (BioVision Inc., Mountain View, Calif.). Viable cells metabolize the WST-8 reagent which results in a color change in their growth medium. This color change can be quantified via a plate reader to obtain % cellular viability.

The cell viability results are presented in Table 3 as normalized cellular proliferation (%). When cellular viability approaches values below 90% viability at PEG amine concentrations between 5 and 1 mg/mL, there exists a strong possibility that the PEG amine may be toxic toward primary endothelial cells. As can be seen from the data in Table 3, P4-10-2 (Example 4) and P8-10-1 (Example 6, Comparative) remained at or above 100% viability at all concentrations tested. In contrast, P8-10-2 (Example 5, Comparative) began to become toxic at a concentration between 0.5 and 1 mg/mL.

TABLE 3
Cell Viability Results from NIH3T3 Fibroblast Assay

| | | PEG Amine | Normalized |
|---|---|---|---|
| | | Concentration | Cellular |
| Example | PEG Amine | (mg/mL) | Proliferation (%) |
| 4 | P4-10-2 | 0.1 | 103 ± 2 |
| | | 0.5 | 111 ± 5 |
| | | 1.0 | 100 ± 6 |
| | | 2.5 | 115 ± 3 |
| | | 5.0 | 124 ± 10 |
| | | 10.0 | 102 ± 12 |
| 5, Comparative | P8-10-2 | 0.1 | 123 ± 3 |
| | | 0.5 | 98 ± 4 |
| | | 1.0 | 85 ± 4 |
| | | 2.5 | 51 ± 4 |
| | | 5.0 | 38 ± 7 |
| | | 10.0 | 27 ± 11 |
| 6, Comparative | P8-10-1 | 0.1 | 118 ± 13 |
| | | 0.5 | 120 ± 7 |
| | | 1.0 | 129 ± 10 |
| | | 2.5 | 133 ± 15 |
| | | 5.0 | 118 ± 2 |
| | | 10.0 | 117 ± 9 |

### Examples 7-9

### Cytocompatibility of Sealants

### Ex vivo Corneal Endothelial Viability Assay

These Examples demonstrate the cytocompatibility of the sealant disclosed herein in an ex vivo corneal endothelial viability assay. This assay is a stringent test of endothelial cellular viability and is representative of a scenario where sealant enters the anterior chamber during surgery.

Fresh porcine eyes were purchased from SiouxPreme Packing Co. (Sioux Center, Iowa) and were shipped overnight on ice by the supplier. Upon receipt, the porcine eyes were placed on ice followed by immediate dissection. The porcine eyes were dissected using a scalpel to make four equidistant 3 mm incisions into the sclera, approximately 4 mm away from the iris. Dissecting scissors were used to cut into the sclera between each 3 mm incision to completely remove the section of the eye containing the cornea, iris and lens. Tweezers were used to isolate the cornea from the iris and lens; the resultant portion of the eye containing the cornea was denoted as the “corneal endothelial cup”. To the corneal endothelial cup, 200 μL of BSS® (Balanced Salt Solution) was added, followed by addition of 5 μL of the test sealant sample (see Table 4), which was applied using the dual component micro delivery device described in Examples 1-3. The sealant samples were incubated in the corneal endothelial cup at 39° C. for 4 hours. To assay for endothelial cellular viability, the test sealant sample and BSS® were removed from the corneal endothelial cup and 200 μL of 0.5 wt % Janus Green dissolved in BSS® was added. The cells were stained for 2 min, and then washed 3 times by submerging in a beaker containing 150 mL of BSS®. The corneal endothelial cups were imaged under a microscope to determine cell death, indicated by the presence of blue dots. If the test material exhibits more dead cells than the control (cells exposed to buffer solution), then the material fails the assay.

The results of the assay are summarized in Table 4. The results from this assay show that sealants comprising P4-10-2 PEG amine (Example 7) and PEG P8-10-1 PEG amine (Example 9, Comparative) are cytocompatible toward primary endothelial cells, while the sealant comprising P8-10-2 PEG amine (Example 8, Comparative) is toxic toward endothelial cells. Although the sealant comprising P8-10-1 PEG amine passed the ex vivo cytocompatibility test, it was observed that the sealant underwent complete degradation within 1 hour; therefore, it does not meet the required degradation profile for an ophthalmic sealant, specifically, the sealant should be visible at the site for at least 3 days.

TABLE 4
Results of Corneal Endothelial Viability Assay

| | First Aqueous | Second Aqueous | |
|---|---|---|---|
| Example | Solution | solution | Cell Viability |
| 7 | D10-50 | P4-10-2 | Pass |
| | 25 wt % | 30 wt % | |
| 8, Comparative | D10-50 | P8-10-2 | Fail |
| | 20 wt % | 30 wt % | |
| 9, Comparative | D10-50 | P8-10-1 | Pass |
| | 25 wt % | 60 wt % | |

### Example 10

### In Vivo Degradation and Tissue Response of Sealant

This Example demonstrates that the sealant disclosed herein meets the requirements set for in vivo degradation and tissue response for an ophthalmic sealant. The set requirements for an ophthalmic sealant were determined to be 1) the sealant should be visible at the incision site for at least three days, and 2) the incisions in test eyes must show tissue response equal to that of the incisions in control eyes.

In the testing, the sealant formed by mixing a first aqueous solution comprising oxidized dextran D10-50 (20-25 wt %) and a second aqueous solution comprising P4-10-2 PEG amine (30 wt %) was used to seal an incision in the eyes of male New Zealand White Rabbits. Each rabbit was anesthetized, and a 3.2 mm wide clear corneal incision (non-self sealing) was made 2-3 mm from the corneal limbal margin of each eye. Both left and right eyes were incised; however only one of the eyes was treated with the sealant. To avoid sample variability, treatment with the sealant was alternated between the right and left eye for the six rabbits. The untreated eye served as a control. To enable visibility of the sealant sample on the incision, a 4 wt % stock solution of FDC Blue #1 dye was added into the first aqueous solution in a volumetric quantity of 0.5%.

After making the incision, an air bubble (approximately 1 cm in diameter) was placed into the anterior chamber of each eye to maintain appropriate incision alignment and prevent leaking of intraocular fluid from the wound during sealant application, and the corneal surface was wiped dry with a surgical sponge. Once the incision was prepared, a 2-4 μL sample of sealant was applied to the incision using the dual component micro delivery device described in Examples 1-3. The resulting mixture was allowed to gel for approximately one minute.

Macroscopic observations were made daily, to document tissue response, and note inflammation and/or irritation at both the control and test incision sites. Sealant visibility was also noted to track degradation.

Under the conditions of the study, there was no corneal irritation or ocular toxicity associated with application of the test sealant. Healing responses were similar between eyes treated with the test sealant and eyes that were untreated. No sealant was evident macroscopically after the third day. Based on the results of the in vivo assay, the sealant disclosed herein met the performance criteria set for an ophthalmic sealant.

What is claimed is:

1. A kit comprising:
a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and
b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

2. The kit according to claim 1 wherein the first aqueous solution comprises the oxidized dextran at about 20 wt % to about 25 wt %.

3. The kit according to claim 1 wherein the second aqueous solution comprises the 4-arm polyethylene glycol at about 25 wt % to about 40 wt %.

4. The kit according to claim 1 wherein the first aqueous solution comprises the oxidized dextran at about 25 wt % and the second aqueous solution comprises the 4-arm polyethylene glycol at about 30 wt %.

5. The kit according to claim 1 wherein at least one of the first aqueous solution or the second aqueous solution further comprises a colorant.

6. A composition comprising the reaction product of:
a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and
b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

7. The composition according to claim 6 wherein the first aqueous solution comprises the oxidized dextran at about 20 wt % to about 25 wt %.

8. The composition according to claim 6 wherein the second aqueous solution comprises the 4-arm polyethylene glycol at about 25 wt % to about 40 wt %.

9. The composition according to claim 6 wherein the first aqueous solution comprises the oxidized dextran at about 25 wt % and the second aqueous solution comprises the 4-arm polyethylene glycol at about 30 wt %.

10. A method of sealing an ophthalmic wound comprising applying to the wound:
a) a first aqueous solution comprising about 15 wt % to about 30 wt % of an oxidized dextran containing aldehyde groups, said oxidized dextran having a weight-average molecular weight of about 8,500 to about 11,500 Daltons and an equivalent weight per aldehyde group of about 130 to about 165 Daltons; and
b) a second aqueous solution comprising about 15 wt % to about 45 wt % of a 4-arm polyethylene glycol substantially each arm of which has two primary amine groups at its end, wherein said 4-arm polyethylene glycol has a number-average molecular weight of about 9,000 Daltons to about 11,000 Daltons.

11. The method according to claim 10 wherein the first aqueous solution comprises the oxidized dextran at about 20 wt % to about 25 wt %.

12. The method according to claim 10 wherein the second aqueous solution comprises the 4-arm polyethylene glycol at about 25 wt % to about 40 wt %.

13. The method according to claim 10 wherein the first aqueous solution comprises the oxidized dextran at about 25 wt % and the second aqueous solution comprises the 4-arm polyethylene glycol at about 30 wt %.

14. The method according to claim 10 wherein at least one of the first aqueous solution or the second aqueous solution further comprises a colorant.

15. The method of claim 10, wherein solution a) and solution b) are applied in a ratio of solution a) to solution b) of 1:3, 1:2, 1:1.5, 1:1, 1.15:1, or 2:1.

16. The method of claim 15, wherein the applied ratio of solution a) to solution b) is 1:1.

17. The method of claim 10 further comprising applying the first aqueous solution and the second aqueous solution to the wound simultaneously without premixing.

18. The method of claim 10 further comprising combining solution a) and solution b) to form a mixture and applying the mixture to the wound.

19. The method of claim 17, wherein solution a) is combined with solution b) in a ratio of 1:3, 1:2, 1:1.5, 1:1, 1.15:1, or 2:1.
